(19) United States
(12) Patent Application Publication
(10) Pub. No.: US 2016/0194336 A1
(43) Pub. Date: Jul. 7, 2016

(54) P38 MAPK INHIBITORS FOR THE TREATMENT OF INFLAMMATORY DISEASES

(71) Applicant: ALLINKY BIOPHARMA, Madrid (ES)

(72) Inventors: Patricia Gómez, Madrid (ES); Esther Carrasco, Madrid (ES); Pedro Campos, Madrid (ES); Patricia Deleyto, Madrid (ES); Miguel Vega, Madrid (ES); Juan Jesús Gómez-Reino, Madrid (ES); Carmen Conde, Madrid (ES); Oreste Gualillo, Madrid (ES); Juan Jesús Pérez, Madrid (ES); Ángel Messeguer, Madrid (ES)

(21) Appl. No.: 14/902,726

(22) PCT Filed: Dec. 17, 2012
(86) PCT No.: PCT/EP2012/075756

§ 371 Date: Jan. 4, 2016

(51) Int. Cl. C07D 498/04; G01N 33/573; C07K 4/12

(52) U.S. Cl. CPC C07D 498/04; C07K 4/12; G01N 33/573; G01N 2333/91205; G01N 2500/04

(57) ABSTRACT

The present invention provides new p38 mitogen activated protein (MAP) kinase allosteric inhibitors which are useful for the treatment of p38 mediated diseases such as inflammatory diseases, e.g. rheumatoid arthritis, osteoarthritis, psoriatic arthritis, pain, musculoskeletal system inflammation and musculoskeletal system aging. The present invention thus provides compounds for use in a method for treatment of inflammatory diseases, as well as for use in therapy in general, wherein the compound binds to the region composed of amino acids at positions 170-199 of Mitogen-activated protein kinase 14 (Uniprot accession nr Q16539 or SEQ ID No 1) and/or Mitogen-activated protein kinase 11 (Uniprot accession nr Q15759 or SEQ ID No 2), SEQ ID NO. 1 and SEQ ID NO. 2 being the amino acid sequences of MAPK14 (p38cx) and MAPK11 (p38β), respectively. The specific region composed of amino acids at positions 170-199 is herein disclosed as SEQ ID NO. 4 for Mitogen-activated protein kinase 14 and SEQ ID NO. 5 for Mitogen-activated protein kinase 11 and are believed to be new inhibitory binding sites.

### FIELD OF THE INVENTION

The present invention provides new p38 mitogen activated protein (MAP) kinase allosteric inhibitors which are useful for the treatment of p38 mediated diseases such as inflammatory diseases, e.g. rheumatoid arthritis, osteoarthritis, psoriatic arthritis and pain.

### BACKGROUND ART

p38α MAP kinase (MAPK) is an intracellular serine/threonine kinase involved in the regulation of inflammatory cell signals and plays a central role in the regulation of pro-inflammatory cytokine production. Activation of p38α is produced by upstream kinases MKK6 and MKK3. At molecular level, like other protein kinases, p38α is responsible for the transfer of the γ-phosphate form ATP to a range of substrate proteins including the transcription factors ATF2, Elk-1 and MEF2A and downstream kinases like MK2, MK3, PRAK, MNK1/2 and MSK1, modulating their function (Stokoe et al., 1992, EMBO J. 11, 3985-3994).

p38α has been identified as a potential target for anti-inflammatory drugs, and different binding sites for these drugs have been identified (Akella et al., January 2008, Biochim Biophys Acta; 1784(1): 48-55). Yong et al. review different p38 MAPK inhibitors which are under development as potential drugs for the treatment of inflammatory diseases and cancer (Yong et al., 2009, Expert Opin. Investig. Drugs; 18(12)). The majority of the drug candidates have proved to be competitive with ATP, binding to the active site. A few inhibitors have also been found that bind to a site adjacent to the active site. Akella et al. discuss the potential relevance of other binding sites such as the binding sites for D-motifs, FXFP and the Backside site.

For example, WO2008/140066 A2 describes pyridone derivative compounds as inhibitors of p38α MAPK, possessing analgesic, anti-inflammatory and arthritis mutilans action.

EP 1 529 531 A1 describes oxadiazolopyrazine derivatives for the treatment protein kinase-dependent diseases such as inflammatory diseases.

### SUMMARY OF THE INVENTION

It is the object underlying the present invention to provide compounds, compositions and formulations that are useful for the treatment of p38 mediated diseases (excluding tumour growth and metastasis (cancer)), such as inflammatory diseases, particularly chronic inflammatory diseases and sustained oxidative stress conditions, such as rheumatoid arthritis, osteoarthritis, dermatitis, fibrosis, neuritis, psoriasis and psoriatic arthritis as well as pain. Particularly, pain caused by an inflammatory process.

The present invention thus provides compounds for use in a method for treatment of inflammatory diseases, as well as for use in therapy in general, wherein the compound binds to the region composed of amino acids at positions 170-199 of Mitogen-activated protein kinase 14 (Uniprot accession nr Q16539 or SEQ ID No 1) and/or Mitogen-activated protein kinase 11 (Uniprot accession nr Q15759 or SEQ ID No 2), SEQ ID NO.1 and SEQ ID NO.2 being the amino acid sequences of MAPK14(p38α) and MAPK11(p38β), respectively. The specific region composed of amino acids at positions 170-199 is herein disclosed as SEQ ID NO.4 for Mitogen-activated protein kinase 14 and SEQ ID NO.5 for Mitogen-activated protein kinase 11 and are believed to be new inhibitory binding sites. Its three-dimensional structure is available from the Protein Data Bank (PDB entry 2OZA).

A first group of suitable compounds which can be used according to the present invention includes peptides comprising SEQ ID NO. 3 (Uniprot accession nr P49137) and having a length of up to 20 amino acids.

Suitable compounds also include small non-peptidyl molecules that mimic the 3D structure and charge distribution of the peptides of SEQ ID NO. 3.

In particular, the present invention provides compounds of the following formula:

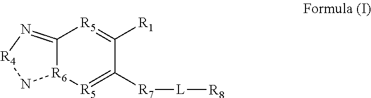

wherein

### R1 is NR2R3, OR2, or CHR2R3;

R2 is a saturated or unsaturated C5-C7-cycloalkyl, saturated or unsaturated C5-C7-heterocycloalkyl, C5-C7-aryl, C5-C7-heteroaryl, a C3-C8-alkyl, C3-C8-alkenyl or C3-C8-alkynyl;
wherein the C5-C7-cycloalkyl, C5-C7-heterocycloalkyl, C5-C7-aryl, C5-C7-heteroaryl, C3-C8-alkyl, C3-C8-alkenyl or C3-C8-alkynyl group is substituted with one or more of a —OR10, —SR10, —NHCOR10, —CONHR10, —COR10, —COOR10, —OCOR10, —NR10R11, —SO₂R10, —SO₂NR10R11, —CF3, —OCF3 or —CN, wherein R10 and R11 are independently of each other hydrogen, C1-C4-alkyl, C1-C4-alkenyl or C1-C4-alkynyl;
R3 is hydrogen or a C1-C4-alkyl, a C2-C4-alkenyl or a C2-C4-alkynyl group;
or wherein R2 and R3 form a five- or six-membered ring;

### R4- - -N- - -R6 is N═N—N, O—N═C or S—N═C;

R5 is independently of each other N or CR11; wherein R11 is hydrogen, OH, or SH;
R7 is an aliphatic chain of the type —NH—, —CH2-, —NH—CH2-, —NH═CH—, —NH—NH—, —NH—CH2-CHR9-, NH—CH═CHR9-, —NH—N═CR9-, —NH—N—CR9-, or

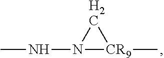

wherein R9 is hydrogen, C1-C4-alkyl, C1-C4-alkenyl or C1-C4-alkynyl;
or wherein R7 is an N-containing saturated or unsaturated C3-C7 heterocycloalkyl, wherein the carbon atom in ortho-position of the atom binding to the bicyclic structure may form a bond with the sulphur or oxygen atom of R5 (in which case H is not present in R5), thereby forming a 5-membered heterocyclic ring, which may be substituted —OH, —SH, —CONH2, —COOH, or —NH2;
L is a linker L, preferably a single covalent bond (so that R7 is linked directly to R8) or a C1 to C3 alkylen group, wherein one of the CH2 groups may be replaced by O, NH, or S;
R8 is saturated or unsaturated C5-C10-cycloalkyl, saturated or unsaturated C5-C10-heterocycloalkyl, C5-C10-aryl, C5-C10-heteroaryl, C5-C10 cycloalkylaryl, C5-C10 heterocycloalkylaryl, C5-C10 cycloalkylheteroaryl, C5-C10 heterocycloalkylheteroaryl, which may be substituted in ortho position with —OH, —SH, —CONH2, —COOH, or —NH2, or a C1-C4 alkyl group; or
which may be substituted in ortho position with an halogen, —OH, —SH, —CONH2, —COOH, —NH2, —OR12, —SR12, or a C1-C4 alkyl group, and which may be substituted at 3 position with an halogen, or—C1-C4 alkyl group, and which may be substituted at 4 position with a halogen, C1-C4 alky group, —OR12 or —SR12 wherein R12 is a C1 to C4 alkyl group.

The present invention also provides methods of treatment using the p38 MAPK inhibitors described herein for the treatment of inflammatory diseases; a method for identifying an inhibitor; and a method of providing an inhibitor.

### FIGURES

FIG. 1: Inhibitory action of the peptides of SEQ ID NO 3 and SEQ ID NO 6 depending on their concentration.

FIG. 2 Inflammatory mediators (IL6, IL8 and MCP-1) in FLS stimulated with TNF-alpha in the presence of compounds 21011_07 and SB (positive control).

FIG. 3: Nitric Oxide production (measuring Nitrate) in chondrocytes stimulated with LPS in the presence of compound 1.

### DETAILED DESCRIPTION

The present invention thus provides compounds for use in therapy such as in a method for treatment of p38 mediated diseases such as inflammatory diseases, wherein the compound binds to the region composed of amino acids at positions 170-199 of SEQ ID NO.1 and/or SEQ ID NO.2, SEQ ID NO.1 and SEQ ID NO.2 being the amino acid sequences of MAPK14(p38α) and MAPK11(p38β), respectively. The compounds of the present invention have preferably an inhibitory effect on the protein of SEQ ID NO.1 and/or SEQ ID NO.2, but not on the mutant R186A or R189A (please refer to Example 2 for the generation of these two mutants) of the protein of SEQ ID NO.1 and/or SEQ ID NO.2.

Suitable compounds can be identified as follows.

The compounds to be used by the present invention are identified by in silico screening and a subsequent binding assay. Starting from a suitable database, such as ZINC (http://zinc.docking.org/), which is a free database of commercially available compounds, several selection criteria should be applied to identify candidates that are then subjected to the subsequent binding assay.

Firstly, the compound must fulfil a pharmacophore structure that is essentially similar to the one fulfilled by the peptides of SEQ ID NO. 3. This peptide was found to fulfil the following pharmacophore, wherein five points are defined as follows:
Point A: a hydrophobic/aromatic moiety;
Point B: a hydrophobic/aromatic moiety;
Point C: a hydrogen acceptor point;
Point D: a hydrogen acceptor point; and
Optional: Point E: a hydrogen donor, wherein the spatial relation between the pharmacophore points are specified by spheres of different radii (r) centered at their Cartesian coordinates: point A (7.99, 34.86 40.16), r=1.5; Point B (12.16; 36.69; 37.15), r=1.6; Point C (7.03, 33.05, 40.27), r=1.2; Point D (9.30, 37.74, 46.62), r=1.5; Point E (12.72, 36.93, 35.072), r=1.6.

These criteria can be used as search criteria in the compound database such as ZINC.

The process for assessing whether a new compound fulfils this pharmacophore requirement is done in a hierarchical manner. In order to make the process more efficient, the pharmacophore is transformed in a set of distance constraints between pairs of chemical moieties: 2.36<d(A,B)<8.56; 4.22<d(B,C)<9.82; 5.51<d(C,D)<10.91; 8.97<d(D,E)<15.17; −0.65<d(A,C)<4.75; 6.84<d(B,D)<13.04; 5.83<d(C,E)<11.43; 4.09<d(A,D)<10.29; −1.03<d(B,E)<5.37; 4.15d(A,E)<10.35. In other words, the spatial relation (in Å) between the pharmacophore points is defined as follows: 2.36<d(A,B)<8.56; 4.22<d(B,C)<9.82; 5.51<d(C,D)<10.91; 8.97<d(D,E)<15.17; 0.65<d(A,C)<4.75; 6.84<d(B,D)<13.04; 5.83<d(C,E)<11.43; 4.09<d(A,D)<10.29; 1.03<d(B,E)<5.37; 4.15<d((A,E)<10.35.

First, the different chemical moieties are identified. For a compound to be considered for a further study, it should exhibit at least four of the requirements of the pharmacophore. Then, the distances between moieties in the molecule are compared with those of the pharmacophore. Finally, if the distances fulfill pharmacophore requirements, molecules are superimposed with the pharmacophore through geometrical superimposition. The root-mean-square-deviation is then used to assess the degree of fulfillment of the pharmacophore.

A second selection rule is that the molecular mass of the compound should be not greater than 500 Dalton. Preferably, the compound complies with the so-called Lipinski's rules, which states that an orally active drug has no more than one violation of the following criteria:
Not more than 5 hydrogen bond donors (nitrogen or oxygen atoms with one or more hydrogen atoms)
Not more than 10 hydrogen acceptors (nitrogen or oxygen atoms)
A molecular mass not greater than 500 Dalton
An octanol-water partition coefficient log P not greater than 5

The hits obtained by in silico screening are then subjected to a p38 binding assay. If the compound shows an inhibitory action on the protein of SEQ ID NO.1 and/or SEQ ID NO.2, but to a lesser extent on the mutant R186A or R189A of the protein of SEQ ID NO.1 and/or SEQ ID NO.2, and if this is achieved in an essentially ATP concentration independent manner, the claimed compound is one that binds to the region composed of amino acids at positions 170-199 of SEQ ID NO.1 and/or SEQ ID NO.2 and can thus be used for therapy according to the present invention.

The compounds have preferably an IC50 of 0.01 nM to 100 μM for inhibiting the MAPK p38 protein activity.

Compounds that bind to the region composed of amino acids at positions 170-199 of SEQ ID NO.1 and/or SEQ ID NO.2 can be structurally rather diverse.

A first group of compounds are peptides comprising SEQ ID NO. 3 and having a length of up to 20 amino acids, preferably up to 10 amino acids. Most preferred are peptides consisting of the amino acid sequence of SEQ ID NO 3 (Tyr Ser Asn His Gly Leu) and or of SEQ ID NO 6 (Phe Tyr Ser Asn His Gly Leu). These peptides fulfil the above stated pharmacophore criteria because the imidazole group of the His and the aliphatic side chain of the Asn act as hydrophobic/aromatic groups (A and B points of the pharmacophore). Moreover, the carbonyl group in the backbone of the Leu fulfils points C and D of the pharmacophore, respectively. Finally the NH moiety of His corresponds to point E of the pharmacophore.

These peptides can be prepared using standard solid or liquid phase peptide synthesis protocols.

A second group of compounds are compounds of the following formula:

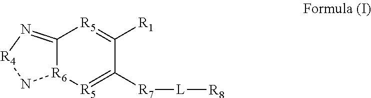

wherein

### R1 is NR2R3, OR2, or CHR2R3;

R2 is a saturated or unsaturated C5-C7-cycloalkyl, saturated or unsaturated C5-C7-heterocycloalkyl, C5-C7-aryl, C5-C7-heteroaryl, a C3-C8-alkyl, C3-C8-alkenyl or C3-C8-alkynyl;
wherein the C5-C7-cycloalkyl, C5-C7-heterocycloalkyl, C5-C7-aryl, C5-C7-heteroaryl, C3-C8-alkyl, C3-C8-alkenyl or C3-C8-alkynyl group is substituted with one or more of a —OR10, —SR10, —NHCOR10, —CONHR10, —COR10, —COOR10, —OCOR10, —NR10R11, —SO₂R10, —SO₂NR10R11, —CF3, —OCF3 or —CN, wherein R10 and R11 are independently of each other hydrogen, C1-C4-alkyl, C1-C4-alkenyl or C1-C4-alkynyl; and
wherein if R1 is NR2R3, the C5-C7-cycloalkyl, C5-C7-heterocycloalkyl, C5-C7-aryl, C5-C7-heteroaryl, C3-C8-alkyl, C3-C8-alkenyl or C3-C8-alkynyl group can be connected to the N via a single bond or NH;
R3 is hydrogen or a C1-C4-alkyl, a C2-C4-alkenyl or a C2-C4-alkynyl group;
or wherein R2 and R3 form a five- or six-membered ring;

### R4- - -N- - -R6 is N═N—N, O—N═C or S—N═C;

R5 is independently of each other N or CR11; wherein R11 is hydrogen, OH, or SH;
R7 is —NH—, —NH—C₆H₄—, —CH2-, —NH—CH2-, —NH═CH—, —NH—NH—, —NH—CH2-CHR9-, —NH—CH═CHR9-, —NH—N═CR9-, —NH—N—CR9-, or

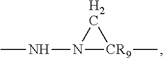

wherein R9 is hydrogen, C1-C4-alkyl, C1-C4-alkenyl or C1-C4-alkynyl;
or wherein R7 is an N-containing saturated or unsaturated C3-C7 heterocycloalkyl, wherein the carbon atom in ortho-position of the atom binding to the bicyclic structure may form a bond with the sulphur or oxygen atom of R5 (in which case H is not present in R5), thereby forming a 5-membered heterocyclic ring, which may be substituted —OH, —SH, —CONH2, —COOH, or —NH2;
L is a linker L, preferably a single covalent bond (so that R7 is linked directly to R8) or a C1 to C3 alkylen group, wherein one of the CH2 groups may be replaced by O, NH, S or CO;
R8 is saturated or unsaturated C5-C10-cycloalkyl, saturated or unsaturated C5-C10-heterocycloalkyl, C5-C10-aryl, C5-C10-heteroaryl, C5-C0 cycloalkylaryl, C5-C10 heterocycloalkylaryl, C5-C10 cycloalkylheteroaryl, C5-C10 heterocycloalkylheteroaryl,
which may be substituted in ortho position with a halogen, —OH, —SH, —CONH2, —COOH, —NH2, —OR12, —SR12, or a C1-C4 alkyl group, and which may be substituted at 3 position with a halogen, or—C1-C4 alkyl group, and which may be substituted at 4 position with a halogen, C1-C4 alky group, —OR12 or —SR12 wherein R12 is a C1 to C4 alkyl group.

R1 is preferably NR2R3.

R2 is preferably a saturated or unsaturated C5-C6-cycloalkyl, saturated or unsaturated C5-C6-heterocycloalkyl, C6-aryl, C5-C6-heteroaryl, a C3-C4-alkyl, C3-C4-alkenyl or C3-C4-alkynyl, which is substituted with one of a —OR10, —SR10, —NHCOR10, —CONHR10, —COR10, —COOR10, —OCOR10, —NR10R11, —SO₂R10, —SO₂NR10OR11, —CF3, —OCF3 or —CN, wherein R10 and R11 are independently of each other hydrogen, C1-C4-alkyl, C1-C4-alkenyl or C1-C4-alkynyl; particularly preferred is a substitution with —OR10 (particularly preferred for R2 being C3-C4-alkyl, C3-C4-alkenyl or C3-C4-alkynyl) or —COOR10 (particularly preferred for R2 being C5-C5-heterocycloalkyl, C6-aryl, C5-C6-heteroaryl; in case of a six-membered ring, the substitution is preferably at position 4, and in case of a five-membered ring, the substitution is preferably at position 2), wherein R10 is hydrogen or C1-C4-alkyl; and

wherein if R1 is NR2R3, the C5-C7-cycloalkyl, C5-C7-heterocycloalkyl, C5-C7-aryl, C5-C7-heteroaryl, C3-C8-alkyl, C3-C8-alkenyl or C3-C8-alkynyl group can be connected to the N via a single bond or NH;

R3 is preferably H.

R4- - -N- - -R6 is preferably N═N—N or O—N═C.

R5 is preferably independently of each other N or CR11, wherein R11 is hydrogen; in another preferred embodiment, one R5 is S and forms a five-membered ring with two carbon atoms of R7 and two atoms of the six-membered ring of the bicyclic core structure, which may be substituted —OH, —SH, —CONH2, —COOH, or —NH2.

R7 is preferably —NH—CH═CHR9-, —NH—N═CR9-, wherein R9 is hydrogen or C1-C4-alkyl, —NH—C₆H₄— or an N-containing saturated or unsaturated C6 heterocycloalkyl.

L is preferably a C1 to C3 alkylen group, wherein one of the CH2 groups may be replaced by O, NH, S or CO;

R8 is preferably C6-C10-aryl, a C6-C10-heteroaryl or a C1-C4 alkyl group.

With regard to these preferred meanings of R1, R2, R3, R4- - -N- - -R6, R5, R7, R8 and L, it is particularly preferred to combine 2 of these preferred meanings, even more preferred being the combination of 3, 4, 5, 6, 7, or 8 of these preferred meanings.

Compounds of Formula I fulfil the pharmacophore requirements as illustrated below:

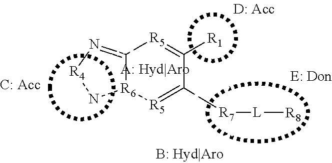

In one preferred —R7-L-R8 is

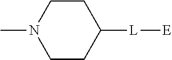

with L being the linker and E being the phenyl ring or the heterocycle.

In another preferred embodiment, —R7-L-R8 is

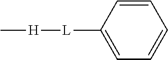

wherein H is an N-containing heterocycle with 5, 6, or 7 ring members, which may be saturated or non-saturated, and which may contain, besides N and CH2/CH, O, S, or N/NH as ring forming elements. Particularly preferred as —R7-L-R8 is

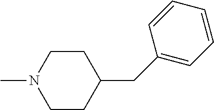

A preferred group of compounds of Formula I are those of Formula II:

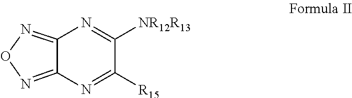

wherein
R12 is hydrogen, or a C1 to C4 alkyl group;
R13 is a ring structure,
the ring structure being an aryl ring which is substituted at positions 3 or 4 with a halogen atom, CF3, OCF3, OR14, NHCOR14, COR14, CONHR14 or COOR14, wherein R14 is a C1 to C8 alkyl group, and which may be substituted with a halogen atom, CF3, or OCF3, a C1 to C4 alkyl group, OR14, NHCOR14, COR14, or COOR14, wherein R14 is a C1 to C8 alkyl group, at the remaining positions;
or an O-, N- and/or S-containing heterocyclic, saturated or non-saturated ring with 5 to 7 ring members which is substituted at 3 or 4 position with a halogen atom, CF3, or OCF3, OR14, NHCOR14, COR14, or COOR14, wherein R14 is a C1 to C8 alkyl group, and which may be substituted with a halogen atom, CF3, OCF3, a C1 to C4 alkyl group, OR14, NHCOR14, COR14, or COOR14, wherein R14 is H or a C1 to C8 alkyl group at the remaining positions; wherein the ring structure or the O-, N- and/or S-containing heterocyclic can be connected to the N via a single bond or NH;
R15 is —NH—C₆H₄—, —NH—N═CH—, or an N-containing heterocycle, with the N binding to the oxazolidinone ring structure, which is linked, via a linker, to either
a C6-C10-aryl or a phenyl ring which might be substituted at 4 position with OR14, NHCOR14, COR14, COOR14, wherein R14 is a C1 to C4 alkyl group, a halogen atom, CF3, or OCF3, and which may be substituted with a C1 to C4 alkyl group, a halogen atom, OR14, NHCOR14, COR14, COOR14, wherein R14 is a C1 to C4 alkyl group a halogen atom CF3, or OCF3 at the remaining positions;
or an O-, N- and/or S-containing heterocyclic, saturated or non-saturated ring with 5 to 7 ring members, and which may be substituted with a C1 to C4 alkyl group, OR14, NHCOR14, COR14, COOR14, wherein R14 is a C1 to C4 alkyl group a halogen atom CF3, or OCF3 at the remaining positions.

The most preferred substituent R13 is

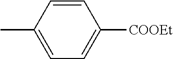

Preferred compounds include the following:

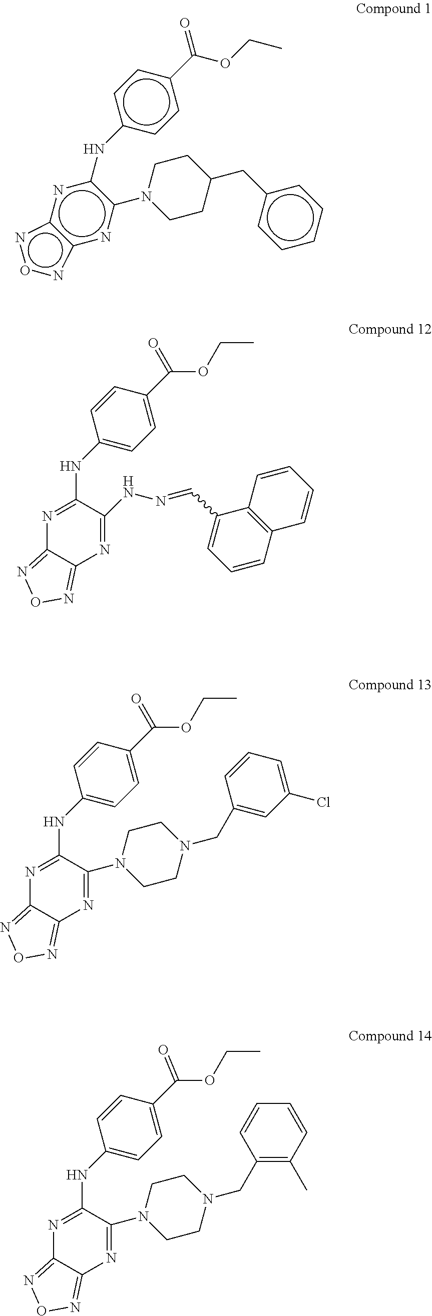

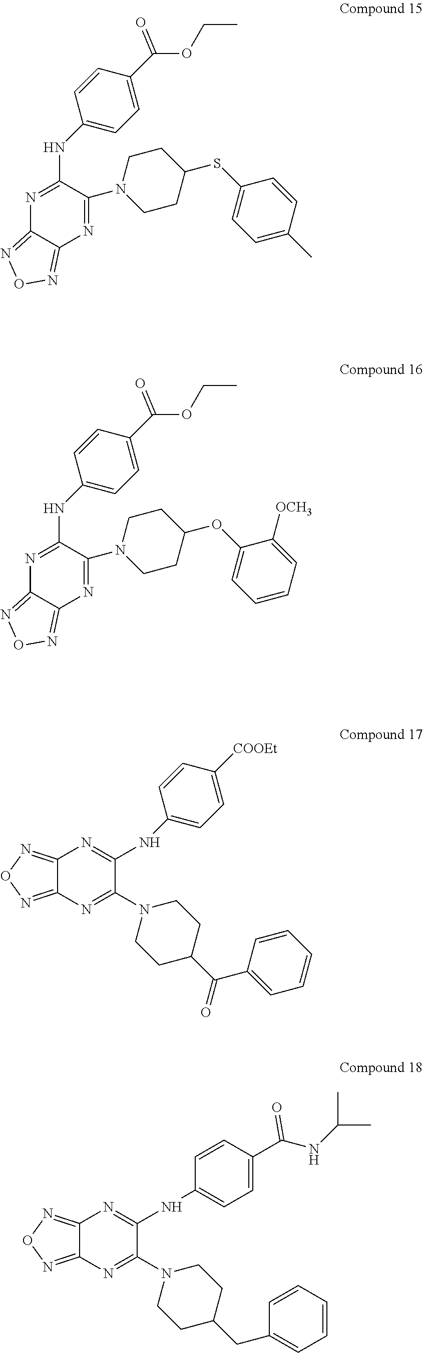 [SMILES: CCOC(=O)c1ccc(Nc2nc3nonc3nc2N2CCC(Sc3ccc(C)cc3)CC2)cc1 ; CCOC(=O)c1ccc(Nc2nc3nonc3nc2N2CCC(Oc3ccccc3OC)CC2)cc1 ; CCOC(=O)c1ccc(Nc2nc3nonc3nc2N2CCC(C(=O)c3ccccc3)CC2)cc1 ; CC(C)NC(=O)c1ccc(Nc2nc3nonc3nc2N2CCC(Cc3ccccc3)CC2)cc1]

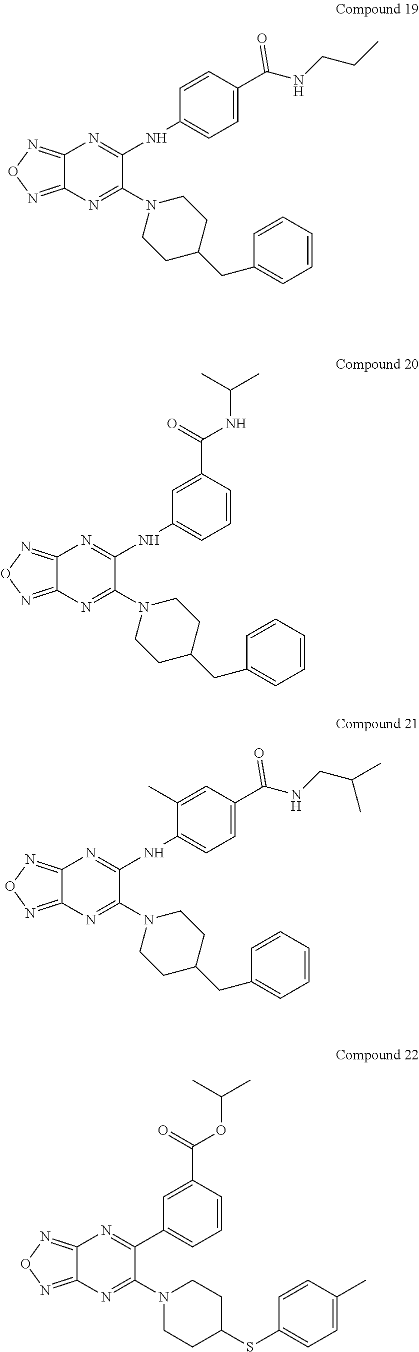 [SMILES: CCCNC(=O)c1ccc(Nc2nc3nonc3nc2N2CCC(Cc3ccccc3)CC2)cc1 ; CC(C)NC(=O)c1cccc(Nc2nc3nonc3nc2N2CCC(Cc3ccccc3)CC2)c1 ; Cc1cc(C(=O)NCC(C)C)ccc1Nc1nc2nonc2nc1N1CCC(Cc2ccccc2)CC1 ; Cc1ccc(SC2CCN(c3nc4nonc4nc3-c3cccc(C(=O)OC(C)C)c3)CC2)cc1]

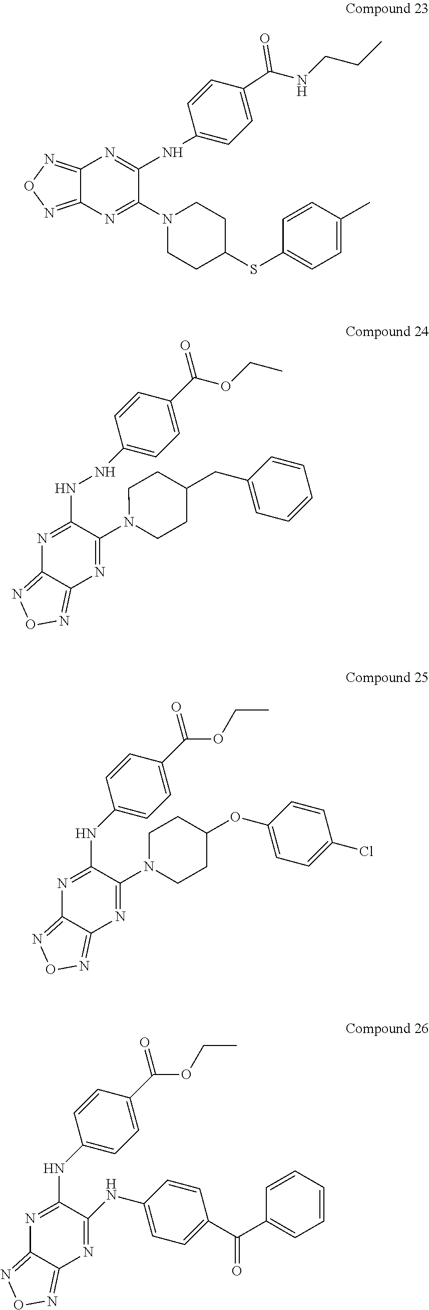 [SMILES: CCCNC(=O)c1ccc(Nc2nc3nonc3nc2N2CCC(Sc3ccc(C)cc3)CC2)cc1 ; CCOC(=O)c1ccc(NNc2nc3nonc3nc2N2CCC(Cc3ccccc3)CC2)cc1 ; CCOC(=O)c1ccc(Nc2nc3nonc3nc2N2CCC(Oc3ccc(Cl)cc3)CC2)cc1 ; CCOC(=O)c1ccc(Nc2nc3nonc3nc2Nc2ccc(C(=O)c3ccccc3)cc2)cc1]

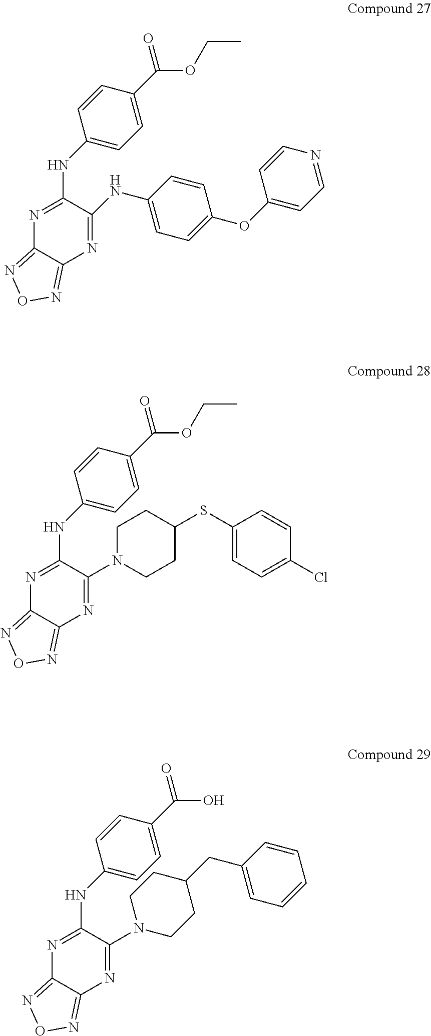 [SMILES: CCOC(=O)c1ccc(Nc2nc3nonc3nc2Nc2ccc(Oc3ccncc3)cc2)cc1 ; CCOC(=O)c1ccc(Nc2nc3nonc3nc2N2CCC(Sc3ccc(Cl)cc3)CC2)cc1 ; O=C(O)c1ccc(Nc2nc3nonc3nc2N2CCC(Cc3ccccc3)CC2)cc1]

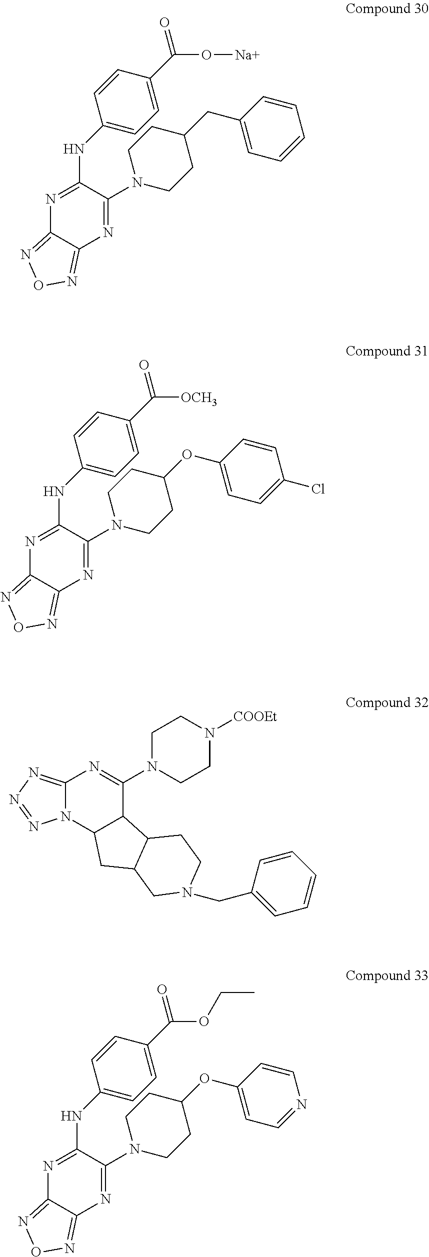

The present invention also provides the use of the compounds of formula I for therapy, such as for the treatment of p38 mediated diseases and disorders, such as inflammatory diseases and disorders.

The compounds of Formula I can be prepared essentially as described by Fernandez et al., 2002, Tetrahedron Letters 43, 4741-4745; Starchenkov et al., Chemistry of Heterocyclic Compounds 1997, 33(19), 1219-1233; and Khim. Geterotskil. Soedin. 1997, 1402-1416. Compound 1 is for example available from Akos GmbH (reference AKOS001630569; http://www.akosgmbh.eu).

Other inhibitors show the following structures:

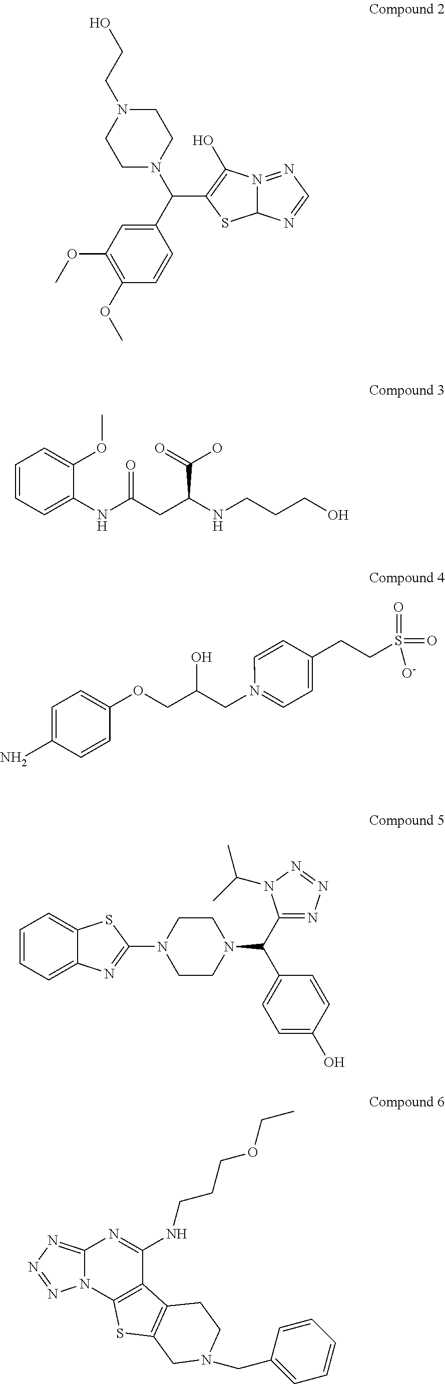

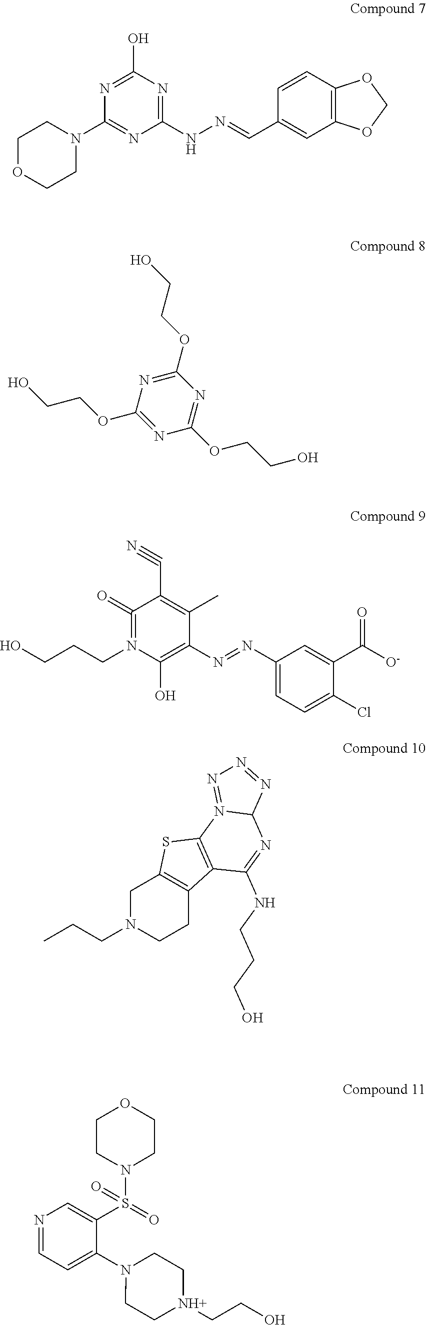

The compounds of the invention include pharmaceutically acceptable salts, esters, amides, and prodrugs thereof, including but not limited to carboxylate salts, amino acid addition salts, esters, amides, and prodrugs of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobsonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like, (See, for example, Berge S. M, et al, “Pharmaceutical Salts,” J. Pharm. Sci., 1977; 66:1-19 which is incorporated herein by reference.)

Accordingly, a further aspect of the present invention includes pharmaceutical compositions comprising as one or more compounds of the invention disclosed above, associated with a pharmaceutically acceptable carrier. For administration, the compounds are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulfuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

Examples of pharmaceutically acceptable, non-toxic esters of the compounds of this invention include C1-C6 alkyl esters, wherein the alkyl group is a straight or branched substituted or unsubstituted, C5-C7 cycloalkyl esters, as well as arylalkyl esters such as benzyl and triphenylmethyl. C1-C4 esters are preferred, such as methyl, ethyl, 2,2,2-trichloroethyl, and tert-butyl. Esters of the compounds of the present invention may be prepared according to conventional methods. Examples of pharmaceutically acceptable, non-toxic amides of the compounds of this invention include amides derived from ammonia, primary C1-C6 alkyl amines and secondary C1-C6 dialkyl amines, wherein the alkyl groups are straight or branched. In the case of secondary amines, the amine may also be in the form, of a 5- or 6-membered heterocycle containing one nitrogen atom. Amides derived from ammonia, C1-C3 alkyl primary amines and C,-C2 dialkyl secondary amines are preferred. Amides of the compounds of the invention may be prepared according to conventional methods.

The term “prodrug” refers to compounds that are rapidly transformed in vivo to yield the parent compound of the above formulae, for example, by hydrolysis in blood. A thorough discussion of prodrugs is provided in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated, by reference.

These compounds can be administered individually or in combination, usually in the form of a pharmaceutical composition. Such compositions are prepared in a manner well known in the pharmaceutical art and comprise at least one active compound.

Accordingly, a further aspect of the present invention includes pharmaceutical compositions comprising as one or more compounds of the invention disclosed above, associated with a pharmaceutically acceptable carrier. For administration, the compounds are ordinarily combined with one or more adjuvants appropriate for the indicated route of administration. The compounds may be admixed with lactose, sucrose, starch powder, cellulose esters of alkanoic acids, stearic acid, talc, magnesium stearate, magnesium oxide, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The compounds and compositions of the present invention are useful for the treatment, prevention or amelioration of one or more symptoms of p38 kinase mediated diseases and disorders, including, but not limited to inflammatory diseases and disorders excluding tumor growth and metastasis (cancer). Particularly the compounds under Markush formula are useful for acute and chronic inflammatory diseases such as rheumatoid arthritis, osteoarthritis, dermatitis, fibrosis, psoriasis, psoriatic arthritis, musculoskeletal system inflammation and musculoskeletal system aging as well as pain, particularly inflammatory pain.

Other p38 mediated diseases for treatment with compounds include Reiter's syndrome, gout, traumatic arthritis, rubella arthritis, acute synovitis, rheumatoid spondylitis, gouty arthritis and other arthritic conditions, sepsis, septic shock, endotoxic shock, gram negative sepsis, toxic shock syndrome, Alzheimer's disease, stroke, ischemic and hemorrhagic stroke, neurotrauma head or spine injury, asthma, adult respiratory distress syndrome, chronic obstructive pulmonary disease, cerebral malaria, meningitis, chronic pulmonary inflammatory disease, silicosis, pulmonary sarcostosis, bone resorption disease, osteoporosis, restenosis, cardiac reperfusion injury, brain and renal reperfusion injury, chronic renal failure, thrombosis, glomerularonephritis, diabetes, non-insulin dependent diabetes, diabetic retinophaty, macular degeneration, graft vs host reaction, allograft rejection, inflammatory bowel disease, Chron's disease, ulcerative colitis, neurodegenerative disease, multiple sclerosis, Amyotrophic lateral sclerosis, diabetic retinopathy, macular degeneration, rhinovirus infection, gingivitis and periodontitis, eczema, contact dermatitis and conjunctivitis.

The present invention also provides a method for identifying compounds that can be used according to the present invention. Hence, the present invention provides a method for screening a compound that binds to the region composed of amino acids at positions 170-199 of SEQ ID NO.1 and/or SEQ ID NO.2, wherein a compound library is subjected to a first binding assay with a protein of SEQ ID NO.1 and/or SEQ ID NO.2, and to a second binding assay with the mutant R186A or R189A of the protein of SEQ ID NO.1 and/or SEQ ID NO.2, and selecting the compound(s) the inhibitory action in the first binding assay is larger than the one in the second binding assay. The present invention also provides a method for providing a compound that binds to the region composed of amino acids at positions 170-199 of SEQ ID NO.1 and/or SEQ ID NO.2, wherein a compound library is subjected to a first binding assay with a protein of SEQ ID NO.1 and/or SEQ ID NO.2, and to a second binding assay with the mutant R186A or R189A of the protein of SEQ ID NO.1 and/or SEQ ID NO.2, and selecting the compound(s) whose inhibitory action in the first binding assay is larger than the one in the second binding assay, and synthesizing the compound or ordering the compound from a provider.

The following examples are intended for the sole purpose of illustrating the present invention.

### EXAMPLES

### Example 1

### Discovery of Novel Binding Site

The rational for considering the specific region composed of amino acids at positions 170-199, herein disclosed as SEQ ID NO.4 for Mitogen-activated protein kinase 14 and SEQ ID NO.5 for Mitogen-activated protein kinase 11 is based in the analysis of the crystal structure of the p38α available from the Protein Data Bank (PDB entry 2OZA). As a proof of concept, Peptide 1 comprising SEQ ID NO.3 belonging to an amino acidic sequence of protein Kinase MK2 was selected for binding to the site.

To analyze the effect of the inhibitory peptides, we performed in vitro kinase assays (IN VITRO PHOSPHORYLATION ASSAY: ADP Quest Method). Purified active p38alpha (25 nM; Invitrogen) was preincubated with the peptides 1 (seven aminoacids) and 2 (six aminoacids) for 10 minutes at 30° C. at concentrations ranging from 4 to 1000 mM in a final volume of 40 ul of kinase buffer (Hepes 15 mM pH 7.4, NaCl 20 mM, EGTA 1 mM, Tween 20 0.02%, MGCl₂ 10 mM, gamma-globulins 0.1%). After that, GST-MEF2A (300 nM) and ATP (100 uM) were added and incubated 30 minutes at 30° C. for the kinase reaction. Phosphorylation was measured as ADP production and ADP was detected using the ADP Quest assay kit (DiscoveRx Corp) following manufacturers instructions. Resultant fluorescence was read with BMG Fluostar microplate reader.

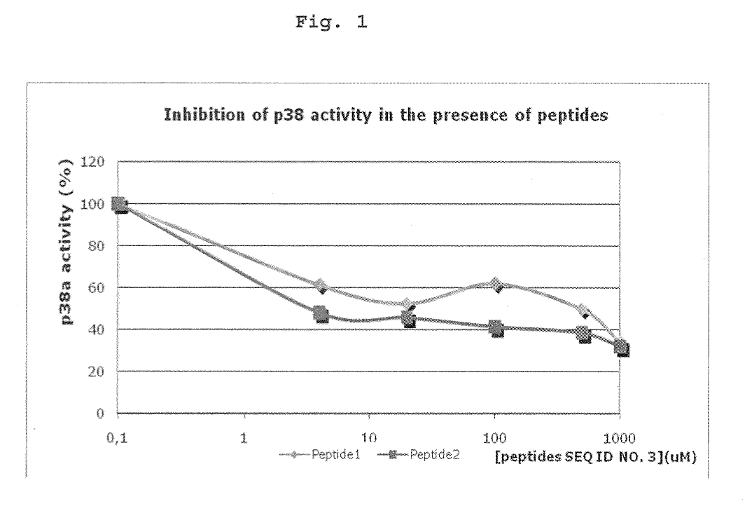

The results are shown in FIG. 1.

### Example 2

### Identification of Compounds Fulfilling Pharmacophore Requirements

In order to identify hits against the novel binding site where the peptides with SEQ ID NO. 3 bind (as described above), an in silico screening of commercially available compounds, such as database ZINC (http://zinc.docking.org), was performed using the pharmacophore defined previously as identification criterion.

The process for assessing if a new compound fulfills pharmacophore requirements is done following a hierarchical procedure:

First, the different chemical moieties are identified. For a compound to be considered for a further study, it should exhibit at least four of the requirements of the pharmacophore. Then, the distances between moieties in the molecule are compared with those of the pharmacophore. Finally, if the distances fulfill pharmacophore requirements, molecules are superimposed with the pharmacophore through geometrical superimposition. The root-mean-square-deviation, between the query features and their matching ligand annotation points, is then used to assess the degree of fulfillment of the pharmacophore.

Hits identified are screening further to assess their drug-like properties using Lipinski's rule of five. The final set of compounds is submitted to a molecular docking procedure. In order to reduce the dimension of the final set, compounds are clustered using the Jarvis-Patrick non-hierarchic method using a 3-point pharmacophore fingerprints as descriptors.

This procedure was followed to identify Compounds 1 to 11 which were purchased and tested in a p38 binding assay.

### Example 3

### Binding Assay

### 3.1. Materials and Methods

To examine the inhibition of the Compounds 1 to 11, in vitro kinase assays were performed. Purified recombinant activated p38alpha (10 nM)(ProQinase) was preincubated 10 minutes at 30° C. with the compound of interest at concentrations between 1 and 100 M in duplicate in a final volume of 30 ul of kinase buffer (Hepes 60 mM pH 7.5, MgCl2 3 mM, MnCl2 3 mM, Sodium Orthovanadate 3 μM, DTT 1.2 mM). After preincubation, peptide substrate (SignalChem #P03-58) and ATP were added to a final concentration of 10 μM and 100 μM respectively, and then incubated 40 minutes at 30° C. for the kinase reaction. Phosphorylation was analyzed with ADP-GloM (Promega #V9101) and emitted luminescence was measured with BMG Fluostar microplate reader.

For p38 cascade kinase reaction, activated p38alpha was substituted by inactive p38alpha (10 nM) (Invitrogen #PV3305) and preincubated 10 minutes at 30° C. with the compound of interest in kinase buffer. After preincubation, MKK6 (ProQinase), ATF2 (ProQinase) and ATP were added to a final concentration of 10 nM, 5 μM and 10 μM respectively, then incubated 40 minutes at 30° C. and analyzed with ADP-Glo™.

### 3.2. Generation of P38αR186A and P38αR189A Mutants

Replacement of specific residues arginine 186 and 189 of p38α (pGEX4T-1-p38α) for alanine was performed with the QuikChange Lightining Site-Directed Mutagenesis Kit (Agilent Technologies). The following oligonucleotides were used for the arginine 186 to alanine substitution: forward primer: 5′-SEQ ID NO 7-3′; reverse primer: 5′-SEQ ID NO 8-3′. Oligonucleotides used for the arginine 189 to alanine substitution were: forward primer: 5′-SEQ ID NO 9-3′; reverse primer: 5′-SEQ ID NO 10-3′. Mutations were validated by sequencing (Genomic Unit, Parque Cientifico de Madrid). E. coli XL10-Gold ultracompentent cells (Agilent Technologies) were transformed with the vector plasmids containing p38αWT and mutants. For the expression of recombinant p38α transformed cells were cultured overnight at 37° C. in 50 ml of fresh LB medium containing ampicillin (100 μg/ml). After that, culture was diluted in fresh LB to A595=0.4 and grown for approximately 1 h at 37° C. until exponential growth and then induced with Isopropyl-β-D-thiogalactopyranoside (IPTG) 1 mM for 5 h. The cells were collected by centrifugation and cell pellet were resuspended in 50 ml of cold lysis buffer: PBS pH 7.4, EDTA 10 mM, PMSF 1 mM, Benzamidine 0.2 mM, DTT 5 mM, Triton X-100 1% and Lysozyme 1 mg/ml. The soluble and insoluble fractions were separated by centrifugation (16,000 g for 20 min). The soluble fraction was incubated for 2 hours with 1 ml of pre-equilibrated Glutathione Sepharose 4B (GE Healthcare) and the resin was extensively washed in a column with 200 ml of binding buffer: PBS, PMSF 1 mM and benzamidine 0.2 mM. Proteins were eluted with 1 ml of Tris-HCl 50 mM pH 8.0 and reduced glutathione 20 mM. 20% glycerol was added to purified proteins and stored at −80° C. Protein concentration and purity was assessed by SDS-PAGE. Kinase activity was measured by ADP-glo kit using MKK6 as activator and ATF2 as substrate. Activity recorded for p38αR186A and p38αR189A were 15.5% and 4.8%, respectively (n=4) related to wild type protein.

### 3.3. Results of the Binding Assay

### 1. Inhibitory Assays Over p38 Cascade Using Different Compounds Predicted by the Docking Algorithm

| | p38a/ATF-2 |
|---|---|
| Compound 2 (10 μM) | 2% |
| Compound 3(100 μM) | 17% |
| Compound 4 (10 μM) | 8% |
| Compound 5 (10 μM) | 28% |
| Compound 6 (10 μM) | 107% |
| Compound 7 (10 μM) | 16% |
| Compound 8(100 μM) | 20% |
| Compound 9 (10 μM) | 9% |
| Compound 10(10 μM) | 43% |
| Compound 11(10 μM) | 1% |

2. Inhibitory Assays Over Four Different MAP Kinase Proteins: Dose Response Achieved with Compound 1

| | p38alpha/ATF-2 | p38beta/ATF-2 |
|---|---|---|
| Compound 1 (100 μM) | 101% | 100% |
| Compound 1 (10 μM) | 88% | 58% |
| SB (10 μM) | 104% | 102% |

3. ATP-Site Competitive Assay: Compound 1 Maintains Inhibition to the Same Level Upon ATP Increase but not SB (n=4) in Cascade p38alpha/ATF-2

| | ATP 10 μM | ATP 100 μM |
|---|---|---|
| Compound 1 (10 μM) | 94% | 92% |
| SB (1 μM) | 81% | 67% |

4. Assay of Compounds In Site-Mutated p38 Over Phosphorylation Cascade: The Compounds Decrease Inhibitory Capacity but SB does not.

| 10 uM | p38alpha wt | R186A | R189A |
|---|---|---|---|
| Compound 1 | 64% | 20% | 25% |
| Compound 12 | 81% | 39% | 14% |
| Compound 13 | 34% | 22% | 14% |
| Compound 14 | 31% | 18% | 17% |
| Compound 15 | 61% | 28% | 20% |
| Compound 16 | 32% | 15% | 17% |
| Compound 17 | 56% | 19% | 13% |
| Compound 18 | 62% | 37% | 33% |
| Compound 19 | 71% | 35% | 32% |
| Compound 20 | 71% | 38% | 22% |
| Compound 21 | 83% | 48% | 38% |
| Compound 22 | 31% | 16% | 6% |
| Compound 23 | 90% | 48% | 26% |
| Compound 24 | 66% | 43% | 39% |
| Compound 25 | 59% | 14% | 30% |
| Compound 26 | 73% | 40% | 28% |
| Compound 27 | 88% | 72% | 47% |
| Compound 28 | 86% | 41% | 27% |
| Compound 29 | 23% | 21% | 6% |
| Compound 30 | 25% | 19% | 9% |
| Compound 31 | 84% | 35% | 27% |
| Compound 32 | 23% | 17% | 9% |
| Compound 33 | 47% | 23% | 19% |
| SB (1 uM) | 60% | 80% | 76% |

SB: An ATP-site competitive inhibitor of p38. It is the standard compound to be used as positive Control.

### Example 4

### Cell-Based Experiments: Results, Materials and Methods

### 4.1. Fibroblast-Like Synoviocytes (FLS)

### 4.1.1. Materials and Methods: Fibroblast-Like Synoviocytes (FLS)

Fibroblast-like synoviocytes (FLS) from patients with rheumatoid arthritis (RA) were obtained at the time of synovectomy or joint replacement. All RA patients fulfilled the American College of Rheumatology 1997 criteria for the diagnosis of RA (Arnett FC y cols. 1988 Arthritis Rheum 31(3):315-24).

Synovial tissue was minced and incubated with 10 μg/ml collagenase in serum-free Dulbecco modified Eagle medium (DMEM; Gibco Invitrogen, Barcelona, Spain) for 3 h at 37° C. After digestion, FLS were filtered through a nylon cell strainer (BD Falcon, Franklin Lakes, USA), washed extensively, and cultured in DMEM supplemented with 10% v/v fetal calf serum, 1% penicillin-streptomycin, and 1% L-glutamine (all reagents from PAA, Laboratories GmbH) in a humidified 5% C02 atmosphere. Adherent cells at 80-90% confluence were trypsinised and diluted at a split ratio of 1:3. FLS from 4 patients and between passages 3 to 8 were used for all experiments.

Enzyme-Linked Immunosorbent Assay (ELISA): RA FLS (1×104 cells/well) were cultured in DMEM 1% FCS in 96 well plates and treated with p-p38 inhibitors for 1 h. After, they were stimulated with TNF-α (10 ng/ml, Sigma Aldrich) and both treatments were present in the culture throughout the experiment. The supernatants were harvested at 18 h after treatment and assayed for IL6, IL8/CXCL8 and monocyte chemoattractant protein (MCP)-1/CCL2 by ELISA according to the manufacturer's instructions (BD Bioscience, San Jose, Calif., USA).

### 4.1.2 Results: Fibroblast-Like Synoviocytes (FLS)

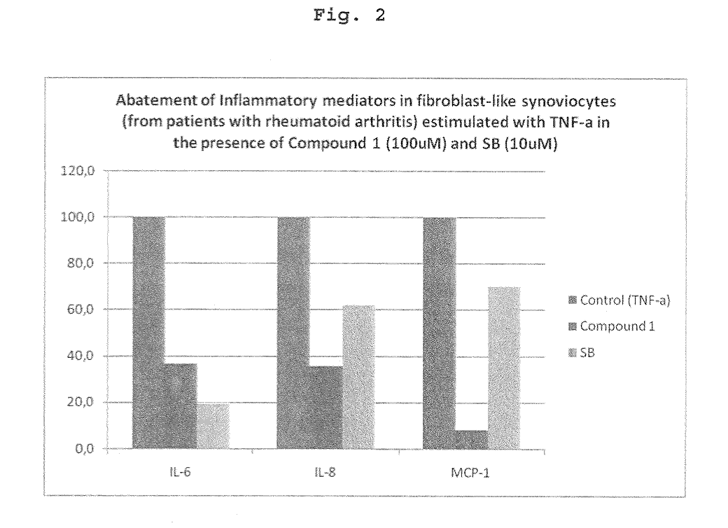

Endpoint: abetment of inflammatory mediators (IL6, IL8 and MCP-1) in FLS stimulated with TNF-alpha in the presence of compounds 1 and SB (positive control). Please also refer to FIG. 2.

| | | | | | | |
|---|---|---|---|---|---|---|
| CONTROL | SH203(%) | R 8(%) | R 6(%) | R 2(%) | Media | ESM |

IL-6

| | | | | | | |
|---|---|---|---|---|---|---|
| TNF-α | 100 | 100 | 100 | 100 | 100.0 | 0.0 |
| 100 μM | 101.4 | 23.5 | 8.1 | 13.8 | 36.7 | 21.8 |
| Comp. 1 | | | | | | |
| SB 10 μM | 57.0 | 17.1 | 35.4 | 11.3 | 19.6 | 4.7 |

IL-8

| | | | | | | |
|---|---|---|---|---|---|---|
| TNF-α | 100 | 100 | 100 | 100 | 100.0 | 0.0 |
| 100 μM | 43.8 | 67.4 | 4.8 | 27.3 | 35.8 | 13.2 |
| Comp. 1 | | | | | | |
| SB 10 μM | 64.4 | 83.9 | 54.1 | 47.8 | 62.1 | 272.7 |

MCP-1

| | | | | | | |
|---|---|---|---|---|---|---|
| TNF-α | 100 | 100 | 100 | 100 | 100.0 | 0.0 |
| 100 μM | 8.8 | 22.5 | 0.0 | 2.1 | 8.4 | 5.1 |
| Comp. 1 | | | | | | |
| SB 10 μM | 77.6 | 76.2 | 63.5 | 61.9 | 70.2 | 78.3 |

Other compounds tested in synoviocytes exerted the following inhibition capacity
% of inflammatory mediators production in the presence of compounds

| [100 uM] | IL6 | IL8 | MCP-1 |
|---|---|---|---|
| TNFa | 100% | 100% | 100% |
| TNFa + Compound 8 | 66.5% | 83.4% | 75.4% |
| TNFa + Compound 9 | 85.4% | 83.2% | 62.5% |
| TNFa + Compound 32 | 52.4% | 34.8% | 22.7% |

### 4.2. Chondrocytes and Macrophages

### 4.2.1 Materials & Methods: Chondrocytes and Macrophages

Cell viability was examined using a colorimetric assay based on the MTT labeling reagent. Briefly, Cells (8×103/well) were seeded in 96-well plates. Assays were performed according to the instructions and protocol provided by the manufacturer (Sigma-Aldrich). After complete adhesion, cells were cultures in serum free conditions (overnight) in order to synchronize cell cycle. Next, cells were incubated with the pertinent drug for 24 or 48 hours with (0.1-50 μM) alone or in combination with 5% FBS medium for 24-48 hours at 37° C. After that, cells were incubated with 10 μl of MTT (5 mg/ml) for 4 hours at 37° C. Then, after dissolving the formazan salt, the spectrophotometric absorbance was measured using a microtiter enzyme-linked immunosorbent assay reader at 550 nm (Multiskan EX; Thermo Labsystems). Data are expressed as % of vitality over unstimulated control. Cell lines used: ATDC5 mouse chondrogenic and J774A1 murine macrophages.

Cell treatments and nitrite assay: ATDC5 cells, as well J774 murine macrophages, with viability greater than 95% evaluated by Trypan blue exclusion method, were cultured in standard conditions in 24-well plates. After 12 h of starvation in serum-free medium, cells were stimulated for 24-48 h with LPS 250 nM, alone or in combination with the drugs. Nitrite accumulation was measured in the culture medium by Griess reaction. Briefly, 100 μl of cell culture medium was mixed with 100 μl of Griess reagent [equal volumes of 1% (wt/vol) sulfanilamide in 5% (vol/vol) phosphoric acid and 0.1% (wt/vol) naphtylethylenediamine-HCl], incubated at room temperature for 10 min, and then the absorbance at 550 nm was measured in a microplate reader (Titertek-Multisca, Labsystem). Fresh culture medium was used as blank in all the experiments. The amount of nitrite in the samples (in micromolar units) was calculated from a sodium nitrite standard curve freshly prepared in culture medium.

### 4.2.2. Results: Chondrocytes and Macrophages

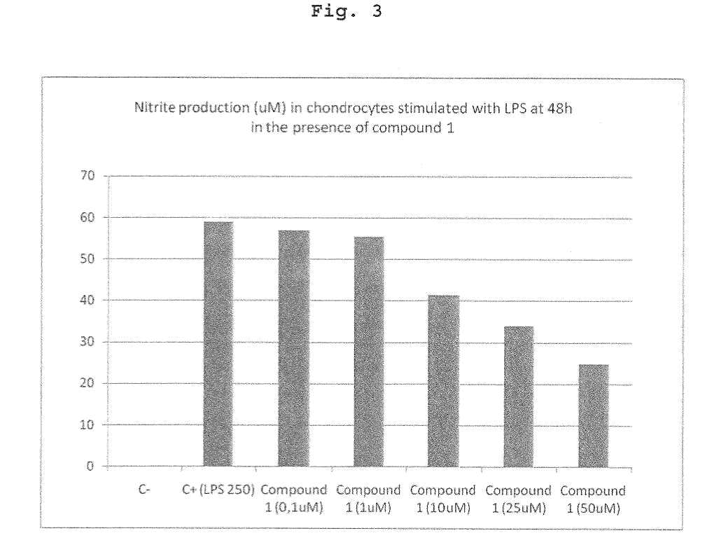

Please refer to FIG. 3.

### 4.3. Human Monocytic Cell Line

### 4.3.1. Materials & Methods: Human Monocytic Cell Line

Inhibition of TNF-α Secretion by a Human Monocytic Cell Line, THP-1.

THP-1 cells, growing in log phase, were collected by centrifugation and resuspended in RPMI 1640 (Sigma Aldrich), to a final cell concentration of 2×106 cells/ml. Cells were plated into 24-well plates (BD Biosciences). Dilutions of compounds in dimethyl sulfoxide (DMSO) were added to the culture to a final concentration M. The final DMSO concentration was 0.5%. The cells ranging from 0.1 to 50 suspensions were preincubated with compounds for 1 h at 37° C. in a 5% C02 humidified atmosphere before the addition of LPS (Sigma Aldrich, L2654) to a final concentration of 2 μg/ml. After that, cells were incubated for 3 h followed by centrifugation to pellet cells. Cell supernatants were stored at 4° C. until Human analysis for TNF-α content. TNF-α levels were determined by ELISA (TNF Biotrak, GE Healthcare) following the manufacturer's directions. The percentage inhibition was calculated for each compound concentration tested, and the IC50 was calculated for each compound.

### 4.3.2. Results:

Abetment of TNF alpha production in monocytes and survival of monocytes with LPS in the presence of compounds 1 and 12 to 31: (see table below)

| | TNF abetment | | | Cell survival | | |
|---|---|---|---|---|---|---|
| | 0.1 uM | 1 uM | 10 uM | 0.1 uM | 1 uM | 10 uM |
| Compound 1 | | 9% | 40% | | 96% | 93% |
| Compound 12 | | −6% | −16% | | 97% | 99% |
| Compound 13 | | 13% | 32% | | 93% | 86% |
| Compound 14 | | | 17% | | | 81% |
| Compound 15 | | 17% | 32% | | 92% | 97% |
| Compound 16 | 16% | 26% | 35% | 88% | 95% | 93% |
| Compound 17 | 21% | 34% | 69% | 99% | 98% | 97% |
| Compound 18 | 1% | 34% | 56% | 90% | 83% | 79% |
| Compound 19 | −47% | −21% | −10% | 103% | 97% | 93% |
| Compound 20 | −9% | 55% | 36% | 101% | 96% | 81% |
| Compound 21 | −15% | 18% | 20% | 98% | 95% | 94% |
| Compound 22 | −2% | 17% | 10% | 97% | 94% | 99% |
| Compound 23 | 26% | 41% | 60% | 96% | 95% | 88% |
| Compound 24 | −23% | 12% | 67% | 104% | 98% | 105% |
| Compound 25 | 12% | 61% | 102% | 88% | 61% | 42% |
| Compound 26 | 27% | 30% | 8% | 86% | 87% | 83% |
| Compound 27 | 28% | 58% | 76% | 103% | 99% | 72% |
| Compound 28 | 17% | 24% | 36% | 88% | 87% | 88% |
| Compound 29 | 7% | 11% | 44% | 103% | 103% | 92% |
| Compound 30 | 12% | 16% | 41% | 99% | 97% | 93% |
| Compound 31 | 20% | 43% | 60% | 101% | 100% | 92% |
| Compound 32 | | | 21% | | | 99% |
| Compound 33 | 21% | 46% | 62% | 100% | 99% | 97% |

1. A compound for use in a method for treatment of p38 mediated diseases or disorders, wherein the compound binds to the region composed of amino acids at positions 170-199 of SEQ ID NO.1 of MAPK14 (p38alpha) and/or SEQ ID NO.2 of MAPK11 (p38beta).

2. A compound for use in the method for treatment of claim 1, wherein the compound:
a) fulfils a pharmacophore structure wherein five points are defined as follows:
Point A: a hydrophobic/aromatic moiety;
Point B: a hydrophobic/aromatic moiety;
Point C: a hydrogen acceptor point;
Point D: a hydrogen acceptor point; and
Optional: Point E: a hydrogen donor,
and wherein the spatial relation between the pharmacophore points are specified by spheres of different radii (r) centered at their Cartesian coordinates: point A (7.99, 34.86 40.16), r=1.5; Point B (12.16; 36.69; 37.15), r=1.6; Point C (7.03, 33.05, 40.27), r=1.2; Point D (9.30, 37.74, 46.62), r=1.5; Point E (12.72, 36.93, 35.072), r=1.6; and
b) Binds to the region composed of amino acids at positions 170-199 of SEQ ID NO.1 of MAPK14 (p38alpha) and/or SEQ ID NO.2 of MAPK11 (p38beta).

3. The compound for use in a method for treatment according to anyone of claims 1 to 2, wherein the compound has an inhibitory effect on the protein of SEQ ID NO.1 and/or of SEQ ID NO.2, but to a lesser extent on the mutant R186A or R189A of the protein of SEQ ID NO.1 and/or of SEQ ID NO.2.

4. The compound for use in a method for treatment according to anyone of claims 1 to 3, for use in a method for treatment or prevention of inflammatory diseases or disorders.

5. The compound for use in a method for treatment according to anyone of claims 1 to 3, for use in a method for treatment or prevention of rheumatoid arthritis, osteoarthritis, psoriatic arthritis, inflammatory pain and musculoskeletal system inflammation.

6. The compound for use in a method for treatment according to one or more of the precedent claims, wherein the compound has an IC50 of 0.01 nM to 100 μM for inhibiting the MAPK p38 protein activity.

7. The compound for use in a method for treatment according to claim 6, wherein the compound has an IC50 of 0.1 nM to 10 μM for inhibiting the MAPK p38 protein activity.

8. The compound for use in a method for treatment according to one or more of the preceding claims, wherein the compound is a peptide of up to 20 amino acids in length and which comprises a hexa- or heptapeptide having anyone of SEQ ID NO 3 or SEQ ID NO 6.

9. The compound for use in a method for treatment according to one or more of preceding claims 1 to 7, wherein the compound is heterocyclic compound of the following general formula: 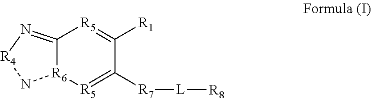
wherein
R1 is NR2R3, OR2, or CHR2R3;
R2 is a saturated or unsaturated C5-C7-cycloalkyl, saturated or unsaturated C5-C7-heterocycloalkyl, C5-C7-aryl, C5-C7-heteroaryl, a C3-C8-alkyl, C3-C8-alkenyl or C3-C8-alkynyl;
wherein the C5-C7-cycloalkyl, C5-C7-heterocycloalkyl, C5-C7-aryl, C5-C7-heteroaryl, C3-C8-alkyl, C3-C8-alkenyl or C3-C8-alkynyl group is substituted with one or more of a —OR10, —SR10, —NHCOR10, —CONHR10, —COR10, —COOR10, —OCOR10, —NR10R11, —SO2R10, —SO2NR10R11, —CF3, —OCF3 or —CN, wherein R10 and R11 are independently of each other hydrogen, C1-C4-alkyl, C1-C4-alkenyl or C1-C4-alkynyl; and
wherein if R1 is NR2R3, the C5-C7-cycloalkyl, C5-C7-heterocycloalkyl, C5-C7-aryl, C5-C7-heteroaryl, C3-C8-alkyl, C3-C8-alkenyl or C3-C8-alkynyl group can be connected to the N via a single bond or NH;
R3 is hydrogen or a C1-C4-alkyl, a C2-C4-alkenyl or a C2-C4-alkynyl group;
or wherein R2 and R3 form a five- or six-membered ring;
R4- - -N- - -R6 is N═N—N, O—N═C or S—N═C;
R5 is independently of each other N or CR11; wherein R11 is hydrogen, OH, or SH;
R7 is —NH—, —NH—C₆H₄—, —CH2-, —NH—CH2-, —NH═CH—, —NH—NH—, —NH—CH2-CHR9-, NH—CH═CHR9-, —NH—N═CR9-, —NH—N—CR9-, or 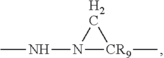
wherein R9 is hydrogen, C1-C4-alkyl, C1-C4-alkenyl or C1-C4-alkynyl;
or wherein R7 is an N-containing saturated or unsaturated C3-C7 heterocycloalkyl, wherein the carbon atom in ortho-position of the atom binding to the bicyclic structure may form a bond with the sulphur or oxygen atom of R5 (in which case H is not present in R5), thereby forming a 5-membered heterocyclic ring, which may be substituted —OH, —SH, —CONH2, —COOH, or —NH2;
L is a linker L, preferably a single covalent bond (so that R7 is linked directly to R8) or a C1 to C3 alkylen group, wherein one of the CH2 groups may be replaced by O, NH, S or CO;
R8 is saturated or unsaturated C5-C10-cycloalkyl, saturated or unsaturated C5-C10-heterocycloalkyl, C5-C10-aryl, C5-C10-heteroaryl, C5-C10 cycloalkylaryl, C5-C10 heterocycloalkylaryl, C5-C10 cycloalkylheteroaryl, C5-C10 heterocycloalkylheteroaryl, which may be substituted in ortho position with a halogen, —OH, —SH, —CONH2, —COOH, —NH2, —OR12, —SR12, or a C1-C4 alkyl group, and which may be substituted at 3 position with a halogen, or—C1-C4 alkyl group, and which may be substituted at 4 position with a halogen, C1-C4 alky group, —OR12 or —SR12; wherein R12 is a C1 to C4 alkyl group;
or a pharmaceutically acceptable salt, ester, amide, and prodrug thereof.

10. The compound for use in a method for treatment according to claim 9, wherein
R1 is NR2R3;
R2 is a saturated or unsaturated C5-C6-cycloalkyl, saturated or unsaturated C5-C6-heterocycloalkyl, C6-aryl, C5-C6-heteroaryl, a C3-C4-alkyl, C3-C4-alkenyl or C3-C4-alkynyl, which is substituted with one of a —OR10, —SR10, —NHCOR10, —CONHR10, —COR10, —COOR10, —OCOR10, —NR10R11, —SO2R10, —SO2NR10R11, —CF3, —OCF3 or —CN, wherein R10 and R11 are independently of each other hydrogen, C1-C4-alkyl, C1-C4-alkenyl or C1-C4-alkynyl; particularly preferred is a substitution with —OR10 (particularly preferred for R2 being C3-C4-alkyl, C3-C4-alkenyl or C3-C4-alkynyl) or —COOR10 (particularly preferred for R2 being C5-C5-heterocycloalkyl, C6-aryl, C5-C6-heteroaryl; in case of a six-membered ring, the substitution is preferably at position 4, and in case of a five-membered ring, the substitution is preferably at position 2), wherein R10 is hydrogen or C1-C4-alkyl;
wherein the saturated or unsaturated C5-C6-cycloalkyl, saturated or unsaturated C5-C6-heterocycloalkyl, C6-aryl, C5-C6-heteroaryl, a C3-C4-alkyl, C3-C4-alkenyl or C3-C4-alkynyl group can be connected to the N via a single bond or NH;
R3 is H.
R4- - -N- - -R6 is N═N—N or O—N═C;
R5 is independently of each other N or CR11, wherein R11 is hydrogen; in another preferred embodiment, one R5 is S and forms a five-membered ring with two carbon atoms of R7 and two atoms of the six-membered ring of the bicyclic core structure, which may be substituted —OH, —SH, —CONH2, —COOH, or —NH2;
R7 is —NH—C₆H₄—, —NH—N═CH—, or an N-containing saturated or unsaturated C6 heterocycloalkyl;
L is a single covalent bond (so that R7 is linked directly to R8) or a C1 to C3 alkylen group, wherein one of the CH2 groups may be replaced by O, NH, S or CO;
R8 is C6-C10-aryl, a C6-C10-heteroaryl or a C1-C4 alkyl group.

11. The compound for use in a method for treatment according to anyone of claim 9 or 10, wherein the compound has the following Formula: 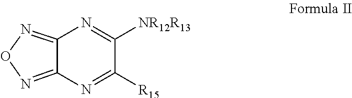
and wherein
R12 is hydrogen, or a C1 to C4 alkyl group;
R13 is a ring structure,
the ring structure being an aryl ring which is substituted at positions 3 or 4 with a halogen atom, CF3, OCF3, OR14, NHCOR14, COR14, CONHR14 or COOR14, wherein R14 is a C1 to C8 alkyl group, and which may be substituted with a halogen atom, CF3, or OCF3, a C1 to C4 alkyl group, OR14, NHCOR14, COR14, or COOR14, wherein R14 is a C1 to C8 alkyl group, at the remaining positions;
or an O-, N- and/or S-containing heterocyclic, saturated or non-saturated ring with 5 to 7 ring members which is substituted at 3 or 4 position with a halogen atom, CF3, or OCF3, OR14, NHCOR14, COR14, or COOR14, wherein R14 is a C1 to C8 alkyl group, and which may be substituted with a halogen atom, CF3, OCF3, a C1 to C4 alkyl group, OR14, NHCOR14,COR14, or COOR14, wherein R14 is H or a C1 to C8 alkyl group at the remaining positions;
wherein the ring structure or the O-, N- and/or S-containing heterocyclic can be connected to the N via a single bond or NH;
R15 is a —NH—C₆H₄—, —NH—N═CH— or an N-containing heterocycle, with the N binding to the oxazolidinone ring structure, which is linked, via a linker, to either
a C6-C10-aryl or a phenyl ring which might be substituted at 4 position with OR14, NHCOR14, COR14, COOR14, wherein R14 is a C1 to C8 alkyl group, a halogen atom, CF3, or OCF3, and which may be substituted with a C1 to C4 alkyl group, a halogen atom, OR14, NHCOR14, COR14, COOR14, wherein R14 is a C1 to C8 alkyl group a halogen atom CF3, or OCF3 at the remaining positions;
or an O-, N- and/or S-containing heterocyclic, saturated or non-saturated ring with 5 to 7 ring members, and which may be substituted with a C1 to C4 alkyl group, OR14, NHCOR14, COR14, COOR14, wherein R14 is a C1 to C8 alkyl group a halogen atom CF3, or OCF3 at the remaining positions;
or a pharmaceutically acceptable salt, ester, amide, and prodrug thereof.

12. Pharmaceutical composition comprising anyone of the following compounds: 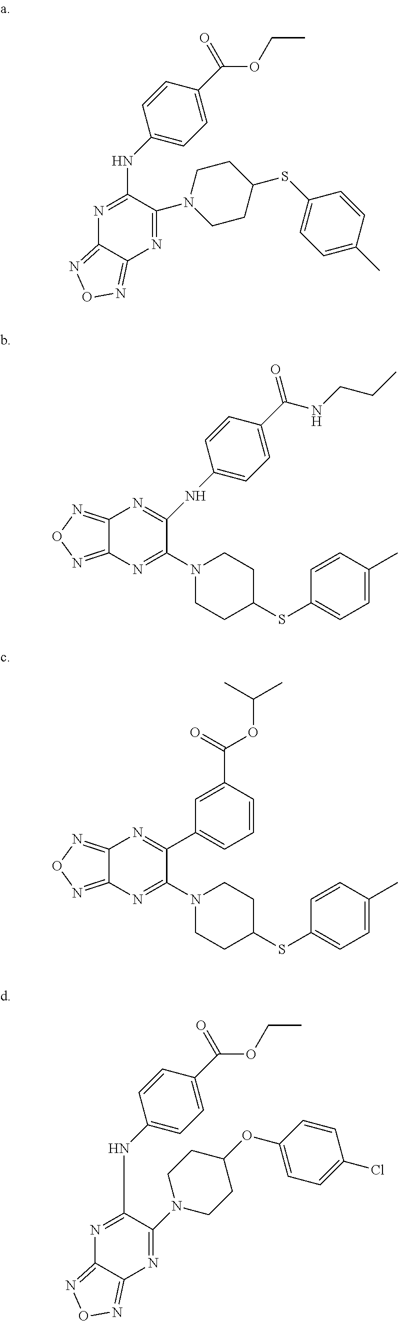 [SMILES: CCOC(=O)c1ccc(Nc2nc3nonc3nc2N2CCC(Sc3ccc(C)cc3)CC2)cc1 ; CCCNC(=O)c1ccc(Nc2nc3nonc3nc2N2CCC(Sc3ccc(C)cc3)CC2)cc1 ; Cc1ccc(SC2CCN(c3nc4nonc4nc3-c3cccc(C(=O)OC(C)C)c3)CC2)cc1 ; CCOC(=O)c1ccc(Nc2nc3nonc3nc2N2CCC(Oc3ccc(Cl)cc3)CC2)cc1] 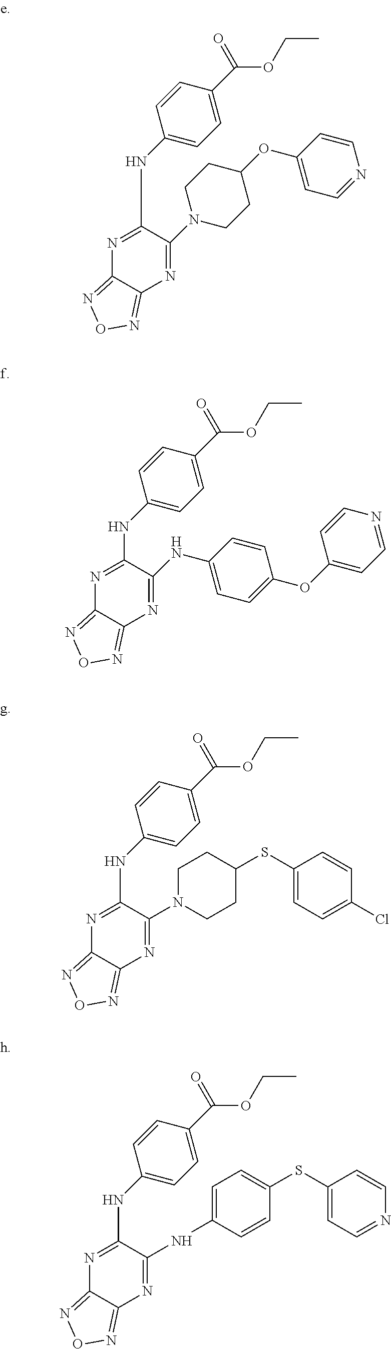 [SMILES: CCOC(=O)c1ccc(Nc2nc3nonc3nc2N2CCC(Oc3ccncc3)CC2)cc1 ; CCOC(=O)c1ccc(Nc2nc3nonc3nc2Nc2ccc(Oc3ccncc3)cc2)cc1 ; CCOC(=O)c1ccc(Nc2nc3nonc3nc2N2CCC(Sc3ccc(Cl)cc3)CC2)cc1 ; CCOC(=O)c1ccc(Nc2nc3nonc3nc2Nc2ccc(Sc3ccncc3)cc2)cc1]
or a pharmaceutically acceptable salt, ester, amide, and prodrug thereof; and
a pharmaceutically acceptable excipient.

13. Method for screening a compound that binds to the region composed of amino acids at positions 170-199 of SEQ ID NO.1 and/or SEQ ID NO.2, wherein a compound library is subjected to a first binding assay with a protein of SEQ ID NO.1 and/or SEQ ID NO.2, and to a second binding assay with the mutant R186A or R189A of the protein of SEQ ID NO.1 and/or SEQ ID NO.2, and selecting the compound(s) the inhibitory action in the first binding assay is larger than the one in the second binding assay.

14. Method for providing a compound that binds to the region composed of amino acids at positions 170-199 of SEQ ID NO.1 and/or SEQ ID NO.2, wherein a compound library is subjected to a first binding assay with a protein of SEQ ID NO.1 and/or SEQ ID NO.2, and to a second binding assay with the mutant R186A or R189A of the protein of SEQ ID NO.1 and/or SEQ ID NO.2, and selecting the compound(s) whose inhibitory action in the first binding assay is larger than the one in the second binding assay, and synthesizing the compound or ordering the compound from a provider.


SEQUENCE LISTING (10 sequences)

SEQ ID NO: 1 - protein, 360 aa, Homo sapiens
  PEPTIDE (1)..(360): Amino acid Sequence of MAPK 14 (p38alpha)
MSQERPTFYRQELNKTIWEVPERYQNLSPVGSGAYGSVCAAFDTKTGLRVAVKKLSRPFQSIIHAKRTYRELRLLKHMKHENVIGLLDVFTPARSLEEFNDVYLVTHLMGADLNNIVKCQKLTDDHVQFLIYQILRGLKYIHSADIIHRDLKPSNLAVNEDCELKILDFGLARHTDDEMTGYVATRWYRAPEIMLNWMHYNQTVDIWSVGCIMAELLTGRTLFPGTDHIDQLKLILRLVGTPGAELLKKISSESARNYIQSLTQMPKMNFANVFIGANPLAVDLLEKMLVLDSDKRITAAQALAHAYFAQYHDPDDEPVADPYDQSFESRDLLIDEWKSLTYDEVISFVPPPLDQEEMES

SEQ ID NO: 2 - protein, 364 aa, Homo sapiens
  PEPTIDE (1)..(364): Peptide Sequence of MAPK 11 (p38beta)
MSGPRAGFYRQELNKTVWEVPQRLQGLRPVGSGAYGSVCSAYDARLRQKVAVKKLSRPFQSLIHARRTYRELRLLKHLKHENVIGLLDVFTPATSIEDFSEVYLVTTLMGADLNNIVKCQALSDEHVQFLVYQLLRGLKYIHSAGIIHRDLKPSNVAVNEDCELRILDFGLARQADEEMTGYVATRWYRAPEIMLNWMHYNQTVDIWSVGCIMAELLQGKALFPGSDYIDQLKRIMEVVGTPSPEVLAKISSEHARTYIQSLPPMPQKDLSSIFRGANPLAIDLLGRMLVLDSDQRVSAAEALAHAYFSQYHDPEDEPEAEPYDESVEAKERTLEEWKELTYQEVLSFKPPEPPKPPGSLEIEQ

SEQ ID NO: 3 - protein, 6 aa, Artificial Sequence
  Peptide Sequence
YSNHGL

SEQ ID NO: 4 - protein, 30 aa, Homo sapiens
  PEPTIDE (1)..(30): Specific region 170-199 of MAPK14 (p38alpha)
GLARHTDDEMTGYVATRWYRAPEIMLNWMH

SEQ ID NO: 5 - protein, 30 aa, Homo sapiens
  PEPTIDE (1)..(30): Specific region 170-199 of MAPK11 (p38beta)
GLARQADEEMTGYVATRWYRAPEIMLNWMH

SEQ ID NO: 6 - protein, 7 aa, Artificial Sequence
  Artificial inhibitory sequence
FYSNHGL

SEQ ID NO: 7 - DNA, 30 bp, Artificial Sequence
  Foward Primer
gctacgtggccactgcgtggtacagggctc

SEQ ID NO: 8 - DNA, 30 bp, Artificial Sequence
  Reverse Primer sequence
agccctgtaccacgcagtggccacgtagcc

SEQ ID NO: 9 - DNA, 32 bp, Artificial Sequence
  Forward primer
cactaggtggtacgcggctcctgagatcatgc

SEQ ID NO: 10 - DNA, 32 bp, Artificial Sequence
  Reverse primer sequence
catgatctcaggagccgcgtaccacctagtgg